(19) United States
(12) Patent Application Publication
(10) Pub. No.: US 2020/0360726 A1
(43) Pub. Date: Nov. 19, 2020

(54) Determination of Tissue Properties for Charged Particle Radiotherapy

(71) Applicant: The Regents of the University of California, Oakland, CA (US)

(72) Inventor: Atchar Sudhyadhom, San Francisco, CA (US)

(73) Assignee: The Regents of the University of California, Oakland, CA (US)

(21) Appl. No.: 16/638,298

(22) PCT Filed: Aug. 13, 2018
(86) PCT No.: PCT/US2018/046513

§ 371 Date: Feb. 11, 2020

Related U.S. Application Data
(60) Provisional application No. 62/544,464, filed on Aug. 11, 2017.

(51) Int. Cl. A61N 5/10; A61B 5/00; A61B 5/145; A61B 5/055; A61B 6/03; G01R 33/56

(52) U.S. Cl. CPC A61N 5/1031; A61B 5/4875; A61B 5/14546; A61B 5/055; A61N 2005/1087; G01R 33/5607; G01R 33/5601; A61N 2005/1034; A61B 6/032

(57) ABSTRACT

The ionization potential of a tissue can be assessed by determining the water content, organic content, and mineral content of the tissue. The methods of determining ionization potential provided here may be implemented by MRI. In one method, ionization potential in soft tissues is achieved by measurement of two tissue parameters: the (1) the amount of water, as a percentage by mass; and (2) the organic hydrogen content of the tissue. Measurement of a third parameter, phosphorous density, allows for calculation of Iₘ in tissues comprising mineralized components. The methods enable more accurate SPR calculation and improved heavy charged particle targeting in radiotherapy.

### CROSS-REFERENCE TO RELATED APPLICATIONS

This application claims the benefit of priority to U.S. Provisional Application Ser. No. 62/544,464 entitled “Determination of Ionization Potential of Tissues by MRI,” filed Aug. 11, 2017, the contents which are hereby incorporated by reference.

### STATEMENT REGARDING FEDERALLY SPONSORED RESEARCH OR DEVELOPMENT

Not Applicable.

### BACKGROUND OF THE INVENTION

Heavy charged particle (HCP) radiotherapy enables the delivery of a high dose of radiation at deep depths while potentially sparing healthy tissue proximal and distal to the target position. Due to the Bragg peak phenomenon, HCPs (most commonly protons) deposit a significant portion of their dose immediately before reaching the end of their range, allowing for concentrated delivery of radiation in the target. However, the efficacy of HCP radiotherapy depends on an accurate prediction of the Bragg peak location in the delivery pathway. The location of the Bragg peak will be in large part determined by the properties of the intervening tissues. Specifically, to effectively target the Bragg peak, the stopping power ratio (SPR) of the intervening tissues between the HCP application point and the target must be calculated.

Unfortunately, current methods of determining SPR are prone to error. A large component of this error is due to uncertainties in the ionization potential (Iₘ), also known as excitation energy. Iₘ is a measure of a tissue's capacity to absorb the energy of HCPs as they pass through the tissue. Current SPR calculation methodologies utilize Iₘ estimations based on certain assumptions regarding the composition of tissues, relying on values for “standard” tissue compositions. However, as known in the art, there is substantial variability between individuals in tissue composition. Furthermore, tissues are highly heterogeneous within the body, such that any model based on standard values will inevitably result in errors due to the heterogeneity of real tissues and their deviations from standard values. As a result, a significant level of uncertainty is inherent in current methods of calculating SPR. As a result, HCP targeting employs large margins in order to account for this uncertainty, resulting in off-target application of radiation.

Accordingly, there is a need in the art for improved methods of determining SPR. Specifically, there is a need in the art for improved methods of calculating Iₘ for tissues surrounding an individual target. If patient-specific and target specific Iₘ values could be calculated with greater accuracy, more accurate SPR determinations could be made and HCP applications could be more precisely directed to targets, with smaller margins and less irradiation of surrounding healthy tissues.

### SUMMARY OF THE INVENTION

Provided herein are novel methods of determining the Iₘ of a tissue. The novel methods of the invention enable more accurate SPR calculation and improved HCP targeting. The novel methods of the invention are based on the discovery that Iₘ can be determined using certain properties of tissue that are measurable by magnetic resonance imaging (MRI). Accordingly, these methods provide the art with a novel means of determining patient-specific and target-specific Iₘ values using clinically available MRI equipment and techniques.

The method of the invention encompasses a determination of Iₘ in soft tissues by measurement of two tissue parameters: the (1) the amount of water, as a percentage by mass; and (2) the content by mass of the organic molecules (for example by the hydrogen content). Using these two readily measured parameters, Iₘ of a tissue volume comprising soft tissue may be determined. Measurement of a third parameter, mineral content (for example by phosphorous or calcium content), allows for calculation of Iₘ in tissues comprising mineralized components.

In one aspect, the scope of the invention encompasses novel methods of calculating Iₘ for a selected volume of tissue. In one aspect, the scope of the invention provides more accurate calculations of SPR and Bragg peak location for HCP treatment planning. In one aspect, the scope of the invention encompasses improved methods of administering HCP treatments to subjects in need thereof In one aspect, the scope of the invention encompasses novel software and hardware elements for estimating Iₘ in tissues and planning HCP treatments.

### BRIEF DESCRIPTION OF THE FIGURES

FIG. 1 is a plot of hydrogen density by mass (h) versus Iₘ (log scale axis) for each molecule studied in Table 1. An exponential fit (Iₘ=(93.23 eV)exp(−3.47 h)) of the organic molecules (plus symbol) is shown as a black line (R²=0.93). Notable exclusions to this fit are: water (open circle), low occurrence amino acids (asterisk), and hydroxyapatite (triangle).

FIG. 2 is a plot of MRI signal versus total hydrogen content for the four chemicals used to create a calibration curve (plus symbol) and oleic acid uncorrected signal (triangle) and oleic acid corrected for T2 decay (asterisk). A linear fit of the calibration chemicals is shown as a black line (R²=0.99).

### DETAILED DESCRIPTION OF THE INVENTION

In first aspect, scope of the invention encompasses methods of determining or estimating tissue properties relevant for proton (and other charged particle therapy) treatments. In one implementation, the general method of the invention encompasses determination of the molecular composition of a sample tissue volume (typically, composed of water, organic (ie. protein, fat, or carbohydrates) and mineral (such as hydroxyapatite) fractions. An assessment of the tissue water content, organic content ((including protein, fat, and carbohydrates), and the mineral content of the sampled tissue volume may be utilized for estimating the Iₘ of the tissue.

In one implementation, the scope of the invention encompasses a simple parameterized method to determine ionization potential, Iₘ, in biological tissues, which allows for computation of subject-specific and site-specific Iₘ at the voxel level using MRI. The method of the invention comprises the determination of mean ionization potential for a selected volume of tissue by three quantities measurable by MRI. The method of the invention calculates Iₘ by assessment of hydrogen (¹H) density and phosphorus (³¹P) density, with two components for hydrogen density, an organic component (lipid, carbohydrate and protein) and a water component.

It will be noted here that certain values utilized in the methods of the invention are enumerated herein. Reference will be made to values being “about” that of the enumerated value. “About” a value, as used herein, will encompass a range of values being up to 10% below than the enumerated value and 10% above the enumerated value. For example, a value of about 10 would encompass the range from 9 to 11. Reference will also be made herein to equations and “equivalents thereof.” The equivalent of an equation means any equation embodying the operative principles of the enumerated equation, regardless of the order of the elements or specific terminology of the equation. Reference will be made herein to methods. It will be understood that the scope of the invention extends to functional equivalents of the enumerated methods, including methods wherein the order of the steps is different, steps are combined, or additional steps are performed therein.

The method of the invention is based on the discovery that Iₘ may be accurately assessed for a given volume of tissue by the assumption that that biological tissues can be segmented into three general components: water (hydrogenous), organic (hydrogenous) and mineralized tissues (calcium/phosphorous rich). In this methodology, the mean ionization potential, Iₘ of molecules is determined by the Bragg additivity rule (BAR) of elemental constituents, as set forth in Equation 1:

$$ln {I}_{m} = ( {\sum }_{i} \frac{{w}_{i} {z}_{i}}{{A}_{i}} ln {I}_{i} ) {( {\sum }_{i} \frac{{w}_{i} {z}_{i}}{{A}_{i}} )}^{- 1} \qquad Equation 1$$

where wᵢ is the fraction by mass of an element (i), Zᵢ is the atomic number for an element (i), and Aᵢ is the atomic mass for an element (i).

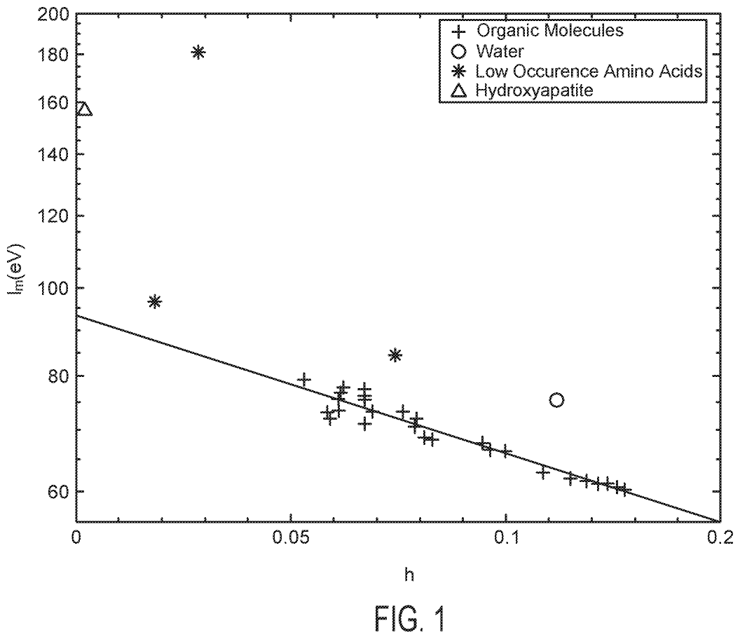

Herein, it is shown that a determination of Iₘ of complex tissues may be made based on Iₘ of water, lipids such as triglycerides, glucose and glycogen, and the 20 common (+2 highly uncommon) amino acids that compose proteins. These highly hydrogenous molecules constitute the vast majority of molecules within tissues. Those molecules are summarized in Table 1 along with elemental compositions and Iₘ as determined by the Bragg additivity rule. Thus, it is demonstrated that for the majority of molecules that comprise human tissues, Iₘ has an exponential relationship with hydrogen content, for example, as depicted in FIG. 1. Notable exceptions to this relationship are: water, three uncommon amino acids (cysteine, methionine and selenocysteine), and hydroxyapatite. The three uncommon amino acids are low in occurrence in human proteins (cysteine 2.3%, methionine 2.13% and selenocysteine <1%) (for example, as known in the art) and can be omitted from the exponential fit of organic molecules.

Thus, the method of the invention can accurately accommodate water and bone mineral compositions (hydroxyapatite [HA]), by a three-component model, the three components being water [H₂O], organic molecules [org], and minerals/hydroxyapatite [HA]), enabling Iₘ determination in a finite sized subset (such as a voxel) of biological tissues, for example, as embodied in Equation 2:

$$ln ( {I}_{v o x e l} ) = ( {\sum }_{m o l e c u l e} \frac{{w}_{m o l e c u l e} {z}_{m o l e c u l e}}{{A}_{m o l e c u l e}} ln ( {I}_{m o l e c u l e} ) ) {( {\sum }_{m o l e c u l e} \frac{{w}_{m o l e c u l e} {z}_{m o l e c u l e}}{{A}_{m o l e c u l e}} )}^{- 1} = [ ( \frac{{w}_{{H}_{2} O} {z}_{{H}_{2} O}}{{A}_{{H}_{2} O}} ln ( {I}_{{H}_{2} O} ) ) + ( {\sum }_{o r g} \frac{{w}_{org} {z}_{org}}{{A}_{org}} ln ( {I}_{o r g} ) ) + ( \frac{{w}_{H A} {z}_{H A}}{{A}_{H A}} ln ( {I}_{H A} ) ) ] {( {\sum }_{m o l e c u l e} \frac{{w}_{m o l e c u l e} {z}_{m o l e c u l e}}{{A}_{m o l e c u l e}} )}^{- 1} \qquad Equation 2$$

where Iᵥₒₓₑₗ is the mean ionization potential for a particular voxel, wᵢ is the fraction by mass of a molecule (i) to all molecules in the voxel such that Σ_{molecule}w_{molecule}=1, Zᵢ is the total atomic number for a molecule (i), Aᵢ is the total atomic mass for a molecule (i), and Iᵢ is the mean ionization potential for a molecule (i). For molecules within the body considered in this study, molecular Z/A values range between 0.47 (selenocysteine) and 0.56 with averages for water (0.56), lipids (0.56), carbohydrates (0.53), amino acids (0.53), and hydroxyapatite (0.50).

In the method of the invention, an assumption that the Z/A ratios for all biological molecules are identical enables simplification of Equation 2 to derive Equation 3:

ln(Iᵥₒₓₑₗ)≈(w_{H}_{₂}_{O} ln(I_{H}_{₂}_{O}))+(Σ_{org} w_{org} ln(I_{org}))+(w_{HA} ln(I_{HA})) Equation 3:

As depicted in FIG. 1, there exists an exponential relationship between I_{org} and hydrogen density by mass from organic molecules, h_{org}, such that I_{org}=A·exp(Bh_{org}) where A and B are constants. For example, in one embodiment, A is between 84 and 102, for example, about 93.23 eV, and B is between −4 and −2.5, for example, being about −3.47. This discovery enables a method of Iₘ determination by further simplifying Equation 3 to Equation 4:

ln(Iᵥₒₓₑₗ)≈(w_{H}_{₂}_{O} ln(I_{H}_{₂}_{O}))+(w_{org,total}[ln A+Bhᵥₒₓₑₗ^{org}])+(w_{HA} ln(I_{HA})) s.t.Σᵢwᵢ=1 Equation 4:

where w_{org,total} is the sum total fractional mass of all organic molecules, A and B are constants that relate hydrogen content in organic molecules to I_{org}, and hᵥₒₓₑₗ^{org} is the total organic molecule hydrogen density by fractional mass in the voxel, A being the intercept of the Iₘ vs. hydrogen density by mass curve and B being the slope of the curve, for example, as in FIG. 1. For soft tissue, the mineral/HA content is considered to be negligible or zero.

Thus, in one method of the invention, Iₘ for each selected volume of soft tissue can be determined as a function of two quantities measurable by MRI: 1) percentage of water/organic materials by mass (w_{H}_{₂}_{O}) and 2) the hydrogen (¹H) content of the organic molecules (h_{org}). For soft tissue, w_{org,total}=(1−w_{H}_{₂}_{O}) and does not need to be independently determined.

For the case of voxels containing mineralized tissues (such as HA), w_{HA} must be determined. ⁴⁰Ca, the 96.941% abundant isotope of calcium, has no magnetic moment and is not amenable to an MRI signal. An alternative to the ⁴⁰Ca signal for mineralized tissues is to image ³¹P. HA has a particular chemical form, Ca₅(PO₄)₃(OH), with a fixed ratio of Ca to P by which P can be used as a proxy for the mineralized tissue (HA) content. The theoretical ratio of the mass content of Ca to P in HA is constant, for example, being estimated in one embodiment as between being about 2.1566. Alternatively, HA or other mineralized tissue content can be determined by dual energy CT or other x-ray based scans.

Thus, the method of the invention enables calculation of Iₘ that accounts for variations in an individual subject's tissue composition at a personal and site-specific level rather than relying the use of “reference” standard human tissue elemental compositions that may not be representative of patient-specific compositions.

In a first aspect, the scope of the invention encompasses a method of estimating Iₘ in a volume of tissue. In one embodiment, the general method of the invention comprises the steps of:
assessing the water content of the tissue volume;
assessing the organic content of the tissue volume;
assessing the hydroxyapatite content of the tissue volume; and
calculating Iₘ based on the relationship between Iₘ and the water content, organic content, and hydroxyapatite content.

The target tissue volume may comprise any volume of tissue found in a subject. The subject may be any animal, for example, a human, a test animal, or a veterinary subject. In one embodiment, the subject is a human subject. In one embodiment, the human subject is a patient in need of an HCP therapy, for example, a subject having cancer, a tumor, or any other neoplastic condition. The tissue volume may comprise the volume of a voxel as implemented by an MRI system. The tissue volume may comprise a volume of any size, for example, from 1-100 mm³. The volume may comprise a potential pathway for the delivery of HCP's to a target region, such as a tumor. The volume may comprise a section of a potential pathway for the delivery of HCP's to a target region, for example wherein multiple volumes across the potential pathway may be determined to arrive at an Iₘ profile for the entire pathway. In one embodiment, Iₘ is profile for target volume, for example, a tumor, and for a volume of tissue surrounding the target representing a potential HCP delivery pathway space.

One step of the process comprises a measurement of water content the tissue volume. Water content may be measured as any quantification of water content, for example, tissue water percentage (w_{H}_{₂}_{O}) by mass. w_{H}_{₂}_{O} may be assessed using any appropriate methodology known in the art. In one embodiment, water percentage is measured or estimated directly. In another embodiment, water percentage is assessed or estimated indirectly, for example, by measurement of a proxy species or parameter which is related to water percentage.

In one embodiment, w_{H}_{₂}_{O} is estimated by the use of MRI data. For example, fat suppression or other water signal measurement techniques may be used to determine w_{H}_{₂}_{O}. Exemplary MRI methods of calculating w_{H}_{₂}_{O} include utilization of data from 2-point Dixon MRI and methods based thereon wherein spin echo images are acquired with fat and water signals in-phase and fat and water signals being out of phase, such water-only or fat suppressed images may be captured to estimate whereby water component can be imaged, Exemplary implementations of the Dixon-2 point method and variations thereof including 1-point, 3-point, multipoint and others, as known in the art. Exemplary methods include those described in: U.S. Pat. No. 9,575,154, entitled “MR imaging using a multi-point Dixon technique,” by Simonetti and Herigault; United States Patent Application Publication Number 2016031423, entitled “Mri with dixon-type water/fat separation with estimation of the main magnetic field variations,” by Eggers; U.S. Pat. No. 8,692,551, “Magnetic resonance imaging water-fat separation method,” by He and Weng; U.S. Pat. No. 7,646,198, entitled “Methods for fat signal suppression in magnetic resonance imaging,” by Bookwalter et al.

In one embodiment, spectrally and spatially selective water-excitation MRI data is used to calculate (w_{H}_{₂}_{O}). For example, methods such as that described by Hore, Solvent suppression in Fourier transform nuclear magnetic resonance. Journal of Magnetic Resonance. 1983, 55: 283-301.

In alternative implementations, the water fraction is assessed by other than MRI. For example, in one embodiment, near-infrared spectroscopy (NIR) data is used to assess water content in the selected tissue volume. In one embodiment, data acquired by acoustic methods is utilized to determine the water content of a target tissue volume. In one embodiment, multi-energy CT may be used to determine tissue compositions, for example, as described in Allessio and MacDonald, Quantitative material characterization from multi-energy photon counting CT, Med. Phys. 2013 March; 40(3):031108.

A second step in the process is the assessment of the content of the organic molecules in the tissue volume, comprising the proteins, carbohydrates, lipids, and other organic molecules present therein. In one implementation, the hydrogen content by mass of the organic molecules in the tissue volume (h_{org}) is used to assess the organic content. Any appropriate method may be used to assess hydrogen content, for example, by MRI methodologies. In one embodiment, h_{org} is measured or estimated directly. In other embodiments, h_{org} is assessed or estimated by measurement of a proxy species or a parameter which is related h_{org}. Exemplary methods of calculating h_{org} include water suppression MRI. Exemplary methods include 2-point Dixon MRI whereby the non-water component can be imaged. In one embodiment, organic component is estimated by measurement of carbons present in the organic fraction, for example by 13C MRI. In one embodiment, the organic component is assessed by multi-energy CT.

In one implementation, w_{H}_{₂}_{O} and h_{org} are determined by performing a proton-density weighted scan and a water/organic ¹H separation scan. The proton-density (PD) weighted scan may comprise any PD-weighted sequence known in the art, for example all gradient and spin echo scans with short TE, long TR, and small flip angles (in the case of gradient echo sequences). The water/organic ¹H separation scan may comprise any water/organic ¹H separation scan sequence known in the art, for example 2-point Dixon, water/fat suppression sequences, FLAIR, and STIR. From these scans, the parameters of w_{H}_{₂}_{O}/w_{org,total} and h_{org} may be obtained.

In one implementation, in the proton-density weighted scan and a water/organic ¹H separation scan, MRI signal (S) may be determined by Equation 5 or an equivalent thereof:

S ∝ ρ_{H}(1−exp(−TR/T₁))exp(−TE/T₂) Equation 5.

where ρ_{H} is the voxel total ¹H content, TR is the repetition time of the pulse sequence, T₁ is the longitudinal relaxation time of the ¹H, TE is the echo time of the pulse sequence, and T₂ is the spin-spin relation time of the ¹H. Technically, a pure ρ_{H} signal requires knowledge and correction of T₁, and T₂, both determinable by MR imaging. To a large extent, a proton (¹H) density-weighted MRI using a spin-echo sequence with TR>>T₁ and TE<<T₂ will produce a reasonable approximation for p_{H} that is not weighted by T₁ or T₂ contrast mechanisms. The ρ_{H} value can be used in conjunction with specific MRI pulse sequences (such as water excitation, water/fat suppression, two-point Dixon, FLAIR, STIR, and SPAIR) to determine the water ¹H versus organic ¹H content. With a proton-density weighted scan and a water/organic ¹H separation scan, all the values for the two parameters of interest for the method of the invention (w_{H}_{₂}_{O}/w_{org,total} and h_{org}) in soft tissue can be attained.

In one implementation, the tissue volume comprises a soft tissue, being a non-mineralized tissue. Exemplary soft tissues include skin, muscle, ligaments, brain, liver, kidney, vascular tissues, digestive tract tissues, tumor, and other non-mineralized tissues. In such tissues, hydroxyapatite content is assumed to be zero and no assessment of hydroxyapatite content is performed.

In the case of tissue volumes comprising mineralized tissues, it will be necessary to account for such materials for the determination of Iₘ. Exemplary mineralized tissues include cortical bone, trabecular, and cancellous bone. In one embodiment, hydroxyapatite fraction of the tissue volume is measured as w_{HA}, comprising the mass percentage of hydroxyapatite in the sample volume. Mineralized tissues primarily comprise hydroxyapatite, having a composition of Ca₅(PO₄)₃(OH). Although ⁴⁰Ca, the predominant form of Ca present in HA, has no MRI detectable signal, the fixed ratio of Ca to P in HA allows quantification of P to be used as a proxy for the HA content. The theoretical ratio of the mass content of Ca to P in HA is 2.1566. For example, a mineral content by mass to phosphorus content by mass (Minerals/P) value in the range of 5.41-5.57 can be used to determine HA fraction by measurement of P. Mill for ³¹P in solid materials may be accomplished by means known in the art, for example, as described by Frey M A, Michaud M, VanHouten J N, Insogna K L, Madri J A and Barrett S E 2012 Phosphorus-31 Mill of hard and soft solids using quadratic echo line-narrowing Proc Natl Acad Sci USA 109 5190-5, Seifert A C, Li C, Raj apakse C S, Bashoor-Zadeh M, Bhagat Y A, Wright A C, Zemel B S, Zavaliangos A and Wehrli F W 2014 Bone mineral (31)P and matrix-bound water densities measured by solid-state (31)P and (1)H Mill NMR Biomed 27 739-48, Seifert A C and Wehrli F W 2016a Erratum to: Solid-State Quantitative 1H and 31P MRI of Cortical Bone in Humans Curr Osteoporos Rep 14 159-61, and Seifert A C and Wehrli F W 2016b Solid-State Quantitative (1)H and (31)P MRI of Cortical Bone in Humans Curr Osteoporos Rep 14 77-86.

In alternative implementations, Ca content can be assessed by CT scan and used as a proxy for HA content, for example by x-ray CT or dual energy x-ray absorption (DEXA) methods.

In the last step of the process, once the parameters of w_{H}_{₂}_{O}/w_{org,total} and h_{org} (if relevant) have been obtained, these values can be used to calculate Iₘ using the mathematical relationship between w_{H}_{₂}_{O}/w_{org,total}, h_{org}, and Iₘ. In one embodiment, the calculation of Iₘ is made by application of Equation 4 or an equivalent thereof, as described above. In one embodiment, a value of A of about 93.23 eV and value of B of about −3.47 is utilized in the implementation of Equation 4.

Determination of SPR. The scope of the invention further encompasses methods of determining SPR using Iₘ values derived by the methods of the invention. Such Iₘ values may be used for the calculation of SPR using any methods known in the art. For example, in one embodiment, the Bethe-Bloch equation, Equation 6 herein, is used to calculate SPR as follows:

$$SPR = {ρ}_{e , water} \frac{ln [ 2 {m}_{e} {c}^{2} {β}^{2} / {I}_{m} ( 1 - {β}^{2} ) ] - {β}^{2}}{ln [ 2 {m}_{e} {c}^{2} {β}^{2} / {I}_{water} ( 1 - {β}^{2} ) ] - {β}^{2}} \qquad Equation 6$$

where of ρ_{e,water} is the electron density normalized to water electron density, mₑ is the electron mass, c is the speed of light, β is the velocity of the proton normalized to c, Iₘ is the mean ionization potential of the medium, and I_{water} is the mean ionization potential of water.

Calculation of SPR for a given volume of tissue requires electron density values be assessed or assumed based on known values. Such electron density values may be determined for the tissue volume using methodologies known in the art, including MRI based methods or methods such as kV SECT, kV DECT and MV CT, as known in the art.

The scope of the invention further encompasses methods of determining Bragg peak location for an HCP beam having specified parameters within a potential delivery pathway, the potential delivery pathway comprising a series of tissue volumes commencing at the external surface of the subject and terminating at the target volume (e.g. tumor location). Bragg peak location may be calculated by any means known in the art wherein Iₘ values calculated by the methods of the invention are utilized.]

Implementations of the Invention. The novel methods described herein for the determination of Iₘ in a sample tissue volume provide the art with a novel and useful means of accomplishing various objectives.

In one aspect, the methods of the invention enable calculation of Iₘ in a selected tissue volume by assessment of the water content of the tissue volume, assessment of the organic component of the tissue volume, assessment of the hydroxyapatite content of the tissue volume, and the relationship between between Iₘ and the water content, organic content, and hydroxyapatite content. In one embodiment, any of the water content of the tissue volume, the organic content of the tissue volume, and the hydroxyapatite content of the tissue volume are assessed by MRI methods. In one embodiment, any two of the water content of the tissue volume, the organic content of the tissue volume, and the hydroxyapatite content of the tissue volume are assessed by MRI methods. In one embodiment, all three of the water content of the tissue volume, the organic content of the tissue volume, and the hydroxyapatite content of the tissue volume are assessed by MRI methods. In one embodiment, the MRI methods comprise the use of Equation 4 or an equivalent thereof. In one embodiment, the application of Equation 4 utilizes an A value of about 93.23 eV and a B value of about −3.47. In one embodiment, the the water content of the tissue volume and the hydrogen content of the organic component of the tissue volume are assessed by a proton-density weighted scan and a water/organic ¹H separation scan. In one embodiment, the hydroxyapatite content of the tissue volume is assessed by MRI methods comprising the measurement of phosphorous-31.

In one embodiment, the selected volume of tissue is a soft tissue and the Iₘ in the selected tissue volume by assessment of the water content of the tissue volume, the organic content of the tissue volume and the relationship between between Iₘ and the water content, and organic content. In one embodiment, the Iₘ of the soft tissue is calculated by the use of Equation 4 or an equivalent thereof. In one embodiment, the application of Equation 4 utilizes an A value of about 93.23 eV and a B value of about −3.47. In one embodiment, the the water content of the soft tissue volume and the organic content of the organic component of the soft tissue volume are assessed by a proton-density weighted scan and a water/organic ¹H separation scan.

In one aspect, the scope of the invention encompasses a method of planning an HCP radiotherapy treatment. In one embodiment, the method of planning an HCP treatment encompasses the steps of assessing Iₘ values along a plurality of potential radiation delivery pathways in a subject and utilizing the assessed Iₘ values in a planning method or planning algorithm to design an HCP administration treatment for delivery of HCPs to a target volume. In one embodiment, the subject is a human subject. In one embodiment the subject is a patient in need of an HCP treatment. In one embodiment, the subject is a cancer patient. In one embodiment, the target volume is a tumor or other neoplasm. In one embodiment, the HCP treatment is a proton beam treatment. In one embodiment, the Iₘ in the selected tissue volumes is calculated by assessment of the water content of the tissue volume, the organic content of the tissue volume and the relationship between between Iₘ and the water content, and organic content. In one embodiment, the Iₘ of the tissue volume is calculated by the use of Equation 4 or an equivalent thereof In one embodiment, the application of Equation 4 utilizes an A value of about 93.23 eV and a B value of about −3.47. In one embodiment, the the water content of the tissue volume and the organic content of the soft tissue volume are assessed by a proton-density weighted scan and a water/organic ¹H separation scan. In one embodiment, the planning method or planning algorithm is utilized to calculate stopping power ratios or equivalent measures of HCP energy dissipation along potential HCP beam paths. In one embodiment, the planning method or planning algorithm is utilized to guide Bragg peak placement at the target volume. In one embodiment, the the planning method or planning algorithm is selected from the group consisting of a Monte Carlo dose calculation, a stoichiometric calibration method, or other proton beam or HCP planning method known in the art.

In one embodiment, the scope of the invention encompasses a method of administering an HCP treatment to a subject in need thereof, comprising the steps of calculating Iₘ values along a plurality of potential radiation delivery pathways in the subject by the methods of the invention, utilizing the calculated Iₘ values to design a treatment plan comprising beam parameters for the delivery of a plurality of beams to a target tissue volume, and the administration of the beams to the subject. In one embodiment, the subject is a cancer patient and the target volume is a tumor or other neoplasm. In one embodiment, the HCP treatment is a proton beam treatment.

In one implementation, the scope of the invention encompasses a tangible or non-transitory electronic storage medium comprising machine-readable instructions (i.e. software) for carrying out a series of operations by a device or group of functionally interconnected devices, such as a processor or general purpose computer. In one embodiment, the electronic storage medium comprises a magnetic or optical storage medium such as a disk, RAM module, FLASH module, or other hardware component.

In one aspect, the tangible storage medium comprises machine-readable instructions for the operation of an MRI system, wherein the instructions cause or enable the MRI system to obtain one or more measurements for calculation of Iₘ values in a selected tissue volume by the methods of the invention. In one embodiment, the one or more measurements for calculation of Iₘ values comprise scans for the assessment of tissue volume water content. In one embodiment, the one or more measurements for calculation of Iₘ values comprise scans for the assessment of organic content of the tissue volume. In one embodiment, the one or more measurements for calculation of Iₘ values comprise scans for the assessment of hydroxyapatite content, for example, by scans for the measurement of phosphorous 31. In one embodiment, the one or more measurements for calculation of Iₘ values comprise performance of a proton-density weighted scan and a water/organic ¹H separation scan.

In one aspect, the tangible storage medium comprises machine-readable instructions for the post-hoc analysis of data obtained by an MRI system for the calculation of Iₘ values in a selected tissue volume by the methods of the invention. In one embodiment, the calculation of Iₘ values in a selected tissue volume is achieved by the performance of Equation 4 or an equivalent thereof. In one embodiment, the tangible storage medium is contained within a general purpose computer or processor. In one embodiment, the tangible storage medium is contained within a component of an MRI system.

In one aspect, the scope of the invention encompasses an MRI system programmed to perform or performing a series of operations for the acquisition of data for calculation of Iₘ values in a selected tissue volume by the methods of the invention. An MRI system, as known in the art, may comprise an assemblage of functionally interconnected devices for the acquisition of MRI data such as T1, T2, and other MRI parameters, for example, comprising a bore, gradient magnets, RF coils, and other components of MRI systems. In one embodiment, the MRI system comprises a combined MRI-proton beam system or other integrated system for the assessment of tissue properties and the delivery of charged particles, e.g., proton beams. In one embodiment, the MRI system is programmed to perform or performs measurements for calculation of the water content, organic content, and/or hydroxyapatite content of a selected tissue volume. In one embodiment, the water content of the tissue volume and the organic of the tissue volume are assessed by a proton-density weighted scan and a water/organic ¹H separation scan. In one embodiment, the hydroxyapatite content of the tissue volume is assessed by MRI methods comprising the measurement of phosphorous-31.

Regarding the various MRI sequences that may be utilized to perform the methods of the invention, it will be understood that the use of appropriate standards can be used to reduce error and improve reproducibility of results between MRI systems. For example, in water quantification and fat suppression techniques, an appropriate fat fraction standard should be utilized, as should a proton density standard for assessing scanner variation when using Proton Density weighted sequences. By use of such standards, sequences may be compared between different subjects, different MR systems, and over different times.

### EXAMPLES

### Example 1. Determination of Mean Ionization Potential Using Magnetic Resonance Imaging

Materials and methods. The Unified Compositions (UC) model. In the unified compositions (UC) model, it is assume that human biological tissues can be segmented into three general components: water (hydrogenous), organic (hydrogenous) and mineralized tissues (calcium/phosphorous rich). In this model, it was determined the mean ionization potential, Iₘ, of molecules by the Bragg additivity rule (BAR) of elemental constituents by Equation 1.

The majority of tissues in humans are composed of five major types of molecules: water, lipids, carbohydrates, proteins, and minerals/hydroxyapatite with the exact proportions dependent on the particular tissue/organ. Five-component models of molecules in the human body have been used previously in the study of the human body and are assumed, in this study, to be sufficient to accurately calculate the mean ionization potential for naturally occurring biological tissues. From the work of Yang et al (Yang M, Virshup G, Clayton J, Zhu X R, Mohan R and Dong L 2010 Theoretical variance analysis of single- and dual-energy computed tomography methods for calculating proton stopping power ratios of biological tissues Phys Med Biol 55 1343-62 and Yang M, Zhu X R, Park P C, Titt U, Mohan R, Virshup G, Clayton J E and Dong L 2012 Comprehensive analysis of proton range uncertainties related to patient stopping-power-ratio estimation using the stoichiometric calibration Phys Med Biol 57 4095-115), it was found that the primary contributor to uncertainties in an individual's tissues was hydrogen content in soft tissues and calcium content in mineralized/bony tissues. Herein was investigated the hydrogen dependence of Iₘ for water, lipids, carbohydrates and proteins by determining Iₘ in water, lipids such as triglycerides, glucose and glycogen, and the 20 common (+2 highly uncommon) amino acids that compose proteins. These highly hydrogenous molecules constitute the vast majority of molecules within human tissues. Those molecules are summarized in Table 1 along with elemental compositions and Iₘ as determined by the Bragg additivity rule. In the majority of molecules that comprise human tissues, Iₘ has an exponential relationship with hydrogen content. That relationship is shown in FIG. 1. Notable exceptions to this relationship are: water, three uncommon amino acids (cysteine, methionine and selenocysteine), and hydroxyapatite. The three uncommon amino acids are low in occurrence in human proteins (cysteine 2.3%, methionine 2.13% and selenocysteine <1%) (Kozlowski, 2017) and were not considered towards the exponential fit of organic molecules. To accurately accommodate water and bone mineral compositions (hydroxyapatite [HA]), Equation 2 is may be utilized for Iₙ, determination in a finite sized subset (such as a voxel) of biological human tissues.

By assuming Z/A ratios for all biological molecules are identical Equation 2 simplified to Equation 3. By the relationship depicted in FIG. 1, I_{org}=A·exp(Bh_{org}) where A and B are constants (A=93.23 eV, B=−3.47 with R²=0.93). This enabled simplification of Equation 3 to Equation 4.

And thus, Iₘ for each voxel in soft tissue can be determined as a function of two quantities measurable by MRI: 1) percentage of water/organic materials by mass (w_{H}_{₂}_{O}) and 2) the hydrogen (¹H) content of the organic molecules (h_{org}). For soft tissue, w_{org,total}=(1−w_{H}_{₂}_{O}) and does not need to be independently determined. For the case of voxels containing mineralized tissues (such as HA), w_{HA} must be determined. As above ³¹P. HA was used as a proxy for the mineralized tissue (HA) content. In Table 2 is show the relationship of P density to total mineral content by percent mass in the dataset of White et al (White D R, Widdowson E M, Woodard H Q and Dickerson J W 1991 The composition of body tissues (II). Fetus to young adult Br J Radiol 64 149-59) to support that a relationship exists between P to mineral content, w_{HA}, in the human body. The theoretical value for mineral content by mass to phosphorus content by mass (Minerals/P) is 5.41 for HA. The average ratio of minerals to P from Table 2 was 5.57 and in line with the theoretical value from HA.

UC Model Evaluation Elemental composition of human tissues and their mean percentage of water, lipid, protein and minerals have been reported in ICRU Report 44 Tables 4.2 and 4.4 (ICRU 1989 Tissue substitutes in radiation dosimetry and measurement (Bethesda, Md., U.S.A.: International Commission on Radiation Units and Measurements and White et al and are regularly cited as “standard” tissue compositions for the purposes of DECT and stoichiometric calibrations and proton stopping power ratio calculations. The accuracy of the UC model was evaluated in these tissues by calculating Iₘ and SPR values by two methods: 1) complete elemental composition (using Bragg's additivity rule [BAR] for each element as in Equation 1 and 2) UC model parameterization using Equation 4 assuming a known electron density for both. Results are tabulated and shown in Table 2 for cortical bone and Table 3 for soft tissue. The stability of the model was also determined in soft tissue to uncertainties in the two parameters by calculating changes in Iₘ due to ±10% changes in w_{H}_{₂}_{O} and h_{org}. Errors of about ±10% were found in these two parameters in the MR measurements in this study and were considered reasonable variations away from actual values for the purposes of the stability analysis. The proton SPR can be approximated by the Bethe-Bloch equation Equation 5. A proton of energy equal to 250 MeV was assumed for all SPR calculations in this work.

Determination of UC model parameters using MRI Spin-echo MR is one of the simplest MR imaging techniques and signals. In the present experiments, signals were attained by Equation 5, as described above.

MRI measurements were carried out on a standard clinical 3T VERIO™ scanner (Siemens Medical Systems GmbH, Erlangen, Germany) using the body coil to minimize signal non-uniformities. The proton density-weighted protocol was implemented using the Siemens spin-echo (referred to below as the spin-echo proton-density [SE PD]) sequence. The following parameters were used: 2D multi-slice acquisition, TR=10,000 ms, TE=4.9 ms, flip angle θ=180°, field of view: (256 mm)², isotropic voxel size: (2 mm)³, slices=64 (interleaved), phase partial Fourier=4/8, and bandwidth=797 Hz. Parameters were chosen to maximize proton-density weighting at reasonable acquisition time of about 11 minutes. T₂ for oleic acid was estimated by acquiring SE PD scans with varying TE (4.9, 10, 20, 40, 80 and 160 ms) and fitting the values to a mono-exponential. A value of 72 ms was determined to be the T₂ relaxation time for oleic acid at room temperature. An additional scan was acquired to separate water from non-water chemicals (referred to below as the turbo spin-echo two-point Dixon [TSE Dixon] sequence). The following parameters were used: 2D multi-slice acquisition, TR=15,000 ms, TE=7.2 ms, flip angle θ=180°, field of view: (256 mm)², isotropic voxel size: (2 mm)³, slices=64 (interleaved), bandwidth=1563 Hz, and turbo factor=3.

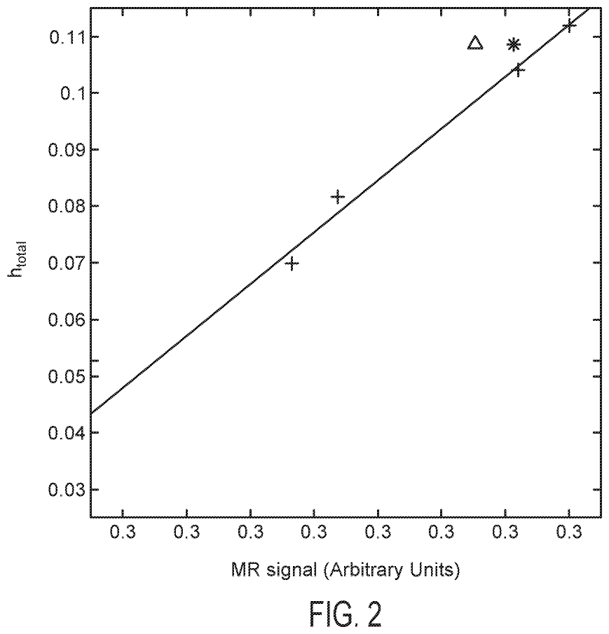

SE PD scans were acquired in a set of chemicals with known compositions to create a calibration curve of total ¹H proton density versus MRI signal (SE PD scan). For calibration curve creation, the following liquid compounds were used: water, acetone, 92% (by volume) isopropyl alcohol, and propargyl alcohol. Chemicals were chosen to span the ¹H content range of organic molecules examined in this study from 7.0% (propargly alcohol) to 11.2% (water). Results for this calibration curve are shown in FIG. 2. As a proof of concept, a fifth compound of known composition was selected, analytical standard grade (>99.0% pure) oleic acid, to be tested but not included in the generation of the calibration curve. A fatty acid was specifically selected for proof of concept testing as adipose tissue (composed of lipids) showed the most potential for instability due to uncertainties in the two parameters of the UC model due to high hydrogen content and low mean ionization potential (relative to water). The mean ionization potential (Iₘ) was calculated using the UC model with its parameters measured by MRI. This Iₘ value was compared to a calculation using only the known chemical composition and Bragg's additivity rule.

Model Evaluation ICRU Report 44 has a number of soft tissue organs in which the mean w_{H}_{₂}_{O} and mean total hydrogen content h are known and are often used as “reference” tissue compositions. These tissues were used in this study to determine the error in modeling all the elements in the calculation of Iₘ (BAR) versus the UC two-parameter model for Iₘ (UC). Results are tabulated in Tables 2 and 3. For cortical bone, the mean percentage difference in Iₘ (BAR) versus Iₘ (UC) is 1.8% with a maximum difference of 2.1%. For the soft tissues studied, the mean percentage difference in Iₘ (BAR) versus Iₘ (UC) is 0.2% with a maximum difference of 1.3% for the eye lens. The high percentage difference for the eye lens organ and cortical bone may in part be due to the high protein content of these organs as amino acids have differing Z/A from water or lipids and individual amino acid compositions in proteins have a larger degree of variation from the molecular fit to Iₘ. Differences in Iₘ result in minimal differences in calculated stopping power ratios (SPR). All calculated SPR from the UC model were within 0.3% of the values calculated by Iₘ (BAR).

The sensitivity of the model was tested in soft tissue by determining the effect of +10% and −10% perturbations on w_{H}_{₂}_{O} and h_{org} on Iₘ (UC) and SPR. Table 4 shows the resulting errors due to a miss determination of w_{H}_{₂}_{O} and h_{org}, respectively, by +/−10%. All values of the perturbed Iₘ (UC) were within 4% of the unperturbed Iₘ (UC) regardless of the type of error. The largest errors were in adipose tissue, which have high fatty acid content. The Iₘ (UC) value perturbations result in minimal errors overall to the SPR. SPR values for adipose tissue were, again, most affected by uncertainties in w_{H}_{₂}_{O} or h_{org} with maximal errors under 0.4% for a 250 MeV proton.

MRI Measurements MR images of the five chemicals, with increasing signal for chemicals with higher hydrogen density. The chemicals with significant MR signal were water and the 92% isopropyl alcohol solution (with 8% water by volume). There was no significant signal from water. A calibration curve (FIG. 2) of normalized MRI signal versus hydrogen density, h, was created for the four hydrogenous chemicals. For the range of molecules (and their respective hydrogen density) considered in this work, there was a linear relationship between MRI signal and total hydrogen density, h. This calibration curve, which did not include oleic acid, was used to estimate the total hydrogen density, h, in oleic acid.

The total hydrogen density content was determined to be 11%, 12%, and 12.1% by MRI, T₂ decay corrected MRI, and calculated by elemental analysis of the composition, respectively, resulting in a 9.7% and 4.2% difference in calculated hydrogen density by MRI and T₂ decay corrected MRI, respectively. TSE Dixon estimated the oleic acid to be 12% water and 88% non-water. These values were used to calculate Iₘ (UC) by MR measurement for oleic acid. Values for Iₘ (BAR) by elemental composition and Iₘ (UC) by MR measurement (using SE PD and TSE Dixon) were 61.1 eV and 65.3 eV, respectively, with a 6.9% difference between the two resulting in an 0.76% difference in calculated SPR for a 250 MeV proton. Iₘ (UC) estimates for oleic acid improve to 63.9 eV when accounting for T₂ decay with a 4.7% difference between Iₘ (UC) and Iₘ (BAR) with a resulting 0.52% difference in calculated SPR for a 250 MeV proton. Recently developed sequences (such as UTE/ZTE) can be further employed to reduce influences on T₁ and T₂ relaxation.

Tests of the methods described herein on tissue compositions demonstrated a high degree of accuracy. Of the tissues theoretically tested, the method of the invention resulted in a mean error in SPR (for a 250 MeV proton) of 0.03% (<0.2% max error) and 0.2% (<0.3% max error) relative to a calculation by all elemental constituents using Bragg's additivity rule for soft tissue and cortical bone, respectively. UC model root-mean-square errors (RMSE) in soft tissue were 0.77% and 0.09% for Iₘ and SPR, respectively. Of particular note is the low UC model error in thyroid which is 0.8% and 0.1% for Iₘ and SPR, respectively. The thyroid tends to have a poorly correlated relationship with effective atomic number (as measured with DECT) relative to other tissues which can lead to a greater uncertainty in the determination of Iₘ and SPR. UC model RMSE in cortical bone were 1.8% and 0.2% for Iₘ and SPR, respectively. SECT stoichiometric method calculation RMSE are typically much larger for SPR calculations. Previous studies showed that the SECT stoichiometric method can result in RMSE of 1.49% with maximum errors at 5.2% for SPR calculations when tested in “reference” tissues. The accuracy of DECT parameterizations for SPR determination have varied reported RMSE from 0.12%-0.28% with maximum errors ranging between 0.39% to 0.98% depending on the type of parameterization used when tested in “reference” tissues. Nearly all of these SECT (stoichiometric) and DECT parameterization methods require parameterization to “reference” human tissues and suffer from increased errors as elemental compositions for particular tissue types deviate away from them. In practice, errors from SECT and DECT calculation of Iₘ and SPR can be much larger.

The UC model was able to account for variations in an individual's tissue composition at a unique level rather than rely on parameterization to “reference” standard human tissue elemental compositions that may not be representative of patient-specific compositions. Compositions of water, fat and protein vary significantly from individual to individual with estimates of 1.2% (1σ) error in SPR for soft tissue being due to deviations in human body tissue composition differences from “reference” standard human tissue compositions. This was demonstrated that mean ionization potential is accurately quantifiable by Mill by the methods described herein.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

TABLE 1
Tabulated chemical compositions for molecules that compose human biological tissues.
Elemental Im values were obtains from ICRU reports for liquids and compounds.

| | | H | C | N | O | P | S | Ca | Se |
|---|---|---|---|---|---|---|---|---|---|
| | | Atomic number (Z) | | | | | | | |
| | | 1 | 6 | 7 | 8 | 15 | 16 | 20 | 34 |
| | | Atomic weight (A) | | | | | | | |
| | | 1.0079 | 12.011 | 14.0067 | 15.9994 | 30.9736 | 32.06 | 40.08 | 78.9 |
| | Chemical | Iₘ (eV) | | | | | | | |
| | Formula | 19.2 | 81 | 82 | 106 | 195.5 | 203.4 | 215.8 | 348 |
| | | | | | | Iₘ (eV) | | | |
| Water | H₂O | 11.2 | | | 88.8 | 75.3 | | | |

Lipids (Ordered by Approximate Prevalence in Humans)

| | | | | | |
|---|---|---|---|---|---|
| Olelic Acid | C₁₈H₃₄O₂ | 12.1 | 76.5 | 11.3 | 61.1 |
| Palmitic | C₁₆H₃₂O₂ | 12.6 | 74.9 | 12.5 | 60.6 |
| Linoleic | C₁₈H₃₂O₂ | 11.5 | 77.1 | 11.4 | 62.0 |
| Palmitoleic | C₁₆H₃₀O₂ | 11.9 | 75.5 | 12.6 | 61.6 |
| Stearic | C₁₈H₃₀O₂ | 12.8 | 76.0 | 11.2 | 60.2 |
| Myristic | C₁₄H₂₈O₂ | 12.4 | 73.6 | 14.0 | 61.1 |
| Linolenic | C₁₈H₃₆O₂ | 10.9 | 77.6 | 11.5 | 62.9 |

Carbohydrates

| | | | | | |
|---|---|---|---|---|---|
| Glycogen | C₆H₁₀O₅ | 6.2 | 44.4 | 49.3 | 77.6 |
| Glucose | C₆H₁₂O₆ | 6.7 | 40.0 | 53.3 | 77.4 |

Amino Acids (Ordered by Approximate Prevalence in Humans)

| | | | | | | |
|---|---|---|---|---|---|---|
| Glycine | C₂H₅NO₂ | 6.7 | 32.0 | 18.7 | 42.6 | 75.5 |
| Proline | C₅H₉NO₂ | 7.9 | 52.2 | 12.2 | 27.8 | 70.5 |
| Alanine | C₃H₇NO₂ | 7.9 | 40.4 | 15.7 | 35.9 | 71.9 |
| Leucine | C₆H₁₃NO₂ | 10.0 | 54.9 | 10.7 | 24.4 | 66.4 |
| Serine | C₃H₇NO₃ | 6.7 | 34.3 | 13.3 | 45.7 | 76.0 |

TABLE 2
Tabulated values for cortical bone elemental and molecular compositions from White
et al (White et al., 1991) in various age humans. Values for Iₘ (BAR) for a calculation
by elemental analysis based off Equation 1, and the three-parameter UC model calculation
for Iₘ using Equation 4. Percentage differences for the two methods (BAR and UC) in Iₘ
and stopping power ratio (SPR) are calculated for a 250 MeV proton.

| | | | | | | | Ca/ | Minerals/ | Iₘ | Iₘ | % | % |
|---|---|---|---|---|---|---|---|---|---|---|---|---|
| | Water | Organics | Minerals | H | P | Ca | P | P | (BAR) | (UC) | Iₘ | SPR |
| Fetus | 41.2 | 25.4 | 33.4 | 6.4 | 6.1 | 13.5 | 2.21 | 5.48 | 94.2 | 95.3 | 1.2% | 0.1% |
| (20 | | | | | | | | | | | | |
| weeks) | | | | | | | | | | | | |
| Fetus | 29.5 | 27.7 | 42.8 | 5.2 | 7.7 | 16.7 | 2.17 | 5.56 | 100.3 | 102.2 | 1.9% | 0.2% |
| (23 | | | | | | | | | | | | |
| weeks) | | | | | | | | | | | | |
| Newborn | 21.1 | 29.3 | 49.6 | 4.4 | 8.5 | 19.4 | 2.28 | 5.84 | 105.0 | 107.3 | 2.1% | 0.3% |
| Infant (3 | 23.2 | 29.8 | 47 | 4.7 | 8.2 | 18.4 | 2.24 | 5.73 | 103.1 | 105.1 | 2.0% | 0.2% |
| months) | | | | | | | | | | | | |
| Child (1 | 20.7 | 31.1 | 48.2 | 4.5 | 8.7 | 19.1 | 2.20 | 5.54 | 104.4 | 106.0 | 1.6% | 0.2% |
| year) | | | | | | | | | | | | |
| Child (5 | 18.9 | 30.8 | 50.3 | 4.3 | 9.3 | 19.8 | 2.13 | 5.41 | 106.0 | 107.6 | 1.5% | 0.2% |
| years) | | | | | | | | | | | | |
| Child | 16.9 | 30.8 | 52.3 | 4 | 9.6 | 20.4 | 2.13 | 5.45 | 107.6 | 109.5 | 1.7% | 0.2% |
| (10 | | | | | | | | | | | | |
| years) | | | | | | | | | | | | |
| Child | 15 | 30.8 | 54.2 | 3.8 | 9.9 | 21 | 2.12 | 5.47 | 108.9 | 111.0 | 1.9% | 0.2% |
| (15 | | | | | | | | | | | | |
| years) | | | | | | | | | | | | |
| Adult | 12.2 | 29.8 | 58 | 3.4 | 10.3 | 22.5 | 2.18 | 5.63 | 112.0 | 114.2 | 2.0% | 0.2% |

TABLE 3
Tabulated values for mean percentage water (w_{H}^{₂}_{O}) by mass and mean
total hydrogen content (h) by mass from ICRU Report #44 for various
soft tissue organs. Calculated values for h_{org}, Iₘ (BAR) for a calculation
based off all elemental components by Equation 1, and the UC two-
parameter model calculation for Iₘ (UC) using Equation 4. Percentage
differences for both methods (BAR and UC) in Iₘ and stopping power
ratio (SPR) are calculated for a 250 MeV proton.

| | Water | H | h_{org} | Iₘ (BAR) | Iₘ (UC) | % Iₘ | % SPR |
|---|---|---|---|---|---|---|---|
| Adipose | 15 | 11.4 | 11.4 | 64.8 | 64.5 | 0.5% | −0.1% |
| Eye lens | 68 | 9.6 | 6.2 | 74.3 | 75.3 | −1.3% | 0.1% |
| Liver | 71 | 10.2 | 7.8 | 74.8 | 74.1 | 0.9% | −0.1% |
| Lung | 78 | 10.3 | 7.1 | 75.2 | 74.7 | 0.6% | −0.1% |
| Muscle | 79 | 10.2 | 6.5 | 74.6 | 75.1 | −0.7% | 0.1% |
| Ovary | 78 | 10.5 | 8.1 | 75 | 74.2 | 1.0% | −0.1% |
| Red | 40 | 10.5 | 10.0 | 69.2 | 69.5 | −0.4% | 0.0% |
| Marrow | | | | | | | |
| Skin | 61.5 | 10 | 8.1 | 73.7 | 73.4 | 0.4% | −0.1% |
| Testis | 81 | 10.6 | 8.1 | 74.7 | 74.4 | 0.5% | −0.1% |
| Thyroid | 75 | 10.44 | 8.0 | 74.7 | 74.1 | 0.8% | −0.1% |

TABLE 4
Tabulated Values for the Percentage Change of Iₘ
using Equation 4 and errors from w_{H}_{₂}₀ and h_{org}
of magnitude +10% and −10%

| | | % Iₘ | % SPR | % Iₘ | % SPR |
|---|---|---|---|---|---|
| | Iₘ | (w_{H}_{₂}_{O}+ | (w_{H}_{₂}_{O}+ | (h_{org}+ | (h_{org}+ |
| | (UC) | 10%) | 10%) | 10%) | 10%) |
| Adipose | 64.5 | −0.3% | 0.03% | 3.3% | −0.4% |
| Eye lens | 75.3 | −0.01% | 0.002% | 0.7% | −0.1% |
| Liver | 74.1 | −0.4% | 0.05% | 0.8% | −0.1% |
| Lung | 74.7 | −0.3% | 0.03% | 0.5% | −0.1% |
| Muscle | 75.1 | −0.1% | 0.01% | 0.5% | −0.1% |
| Ovary | 74.2 | −0.5% | 0.1% | 0.6% | −0.1% |
| Red Marrow | 69.5 | −0.5% | 0.1% | 2.1% | −0.2% |
| Skin | 73.4 | −0.4% | 0.05% | 1.1% | −0.1% |
| Testis | 74.4 | −0.5% | 0.1% | 0.5% | −0.1% |
| Thyroid | 74.1 | −0.5% | 0.1% | 0.7% | −0.1% |

| | % Iₘ | % SPR | % Iₘ | % SPR |
|---|---|---|---|---|
| | (w_{H}_{₂}_{O}− | (w_{H}_{₂}_{O}− | (h_{org}− | (h_{org}− |
| | 10%) | 10%) | 10%) | 10%) |
| Adipose | −3.2% | 0.4% | −3.4% | 0.4% |
| Eye lens | −0.8% | 0.1% | −0.7% | 0.1% |
| Liver | −0.6% | 0.1% | −0.8% | 0.1% |
| Lung | −0.5% | 0.1% | −0.5% | 0.1% |
| Muscle | −0.6% | 0.1% | −0.5% | 0.1% |
| Ovary | −0.3% | 0.0% | −0.6% | 0.1% |
| Red Marrow | −1.7% | 0.2% | −2.1% | 0.2% |
| Skin | −0.8% | 0.1% | −1.1% | 0.1% |
| Testis | −0.2% | 0.0% | −0.5% | 0.1% |
| Thyroid | −0.4% | 0.0% | −0.7% | 0.1% |

What is claimed is:

1. A method of estimating the ionization potential value, Iₘ, for a volume of tissue, comprising the steps of:
assessing the water content of the tissue volume;
assessing the organic content of the tissue volume;
assessing the hydroxyapatite content of the tissue volume; and
calculating Iₘ based on the relationship between Iₘ and the water content, organic content, and hydroxyapatite content of the tissue volume.

2. The method of claim 1, wherein
the volume of tissue comprises tissue in a human subject.

3. The method of claim 1, wherein
the human subject is a patient in need of a HCP therapy.

4. The method of claim 1, wherein
the determination of the water content is made by MRI.

5. The method of claim 5, wherein
the determination of water content is made by fat suppression MRI or a variation thereof.

6. The method of claim 1, wherein
the assessment of water content is made by water excitation MRI.

7. The method of claim 1, wherein
the assessment of the organic content of the tissue volumes is made by MRI.

8. The method of claim 7, wherein
the assessment of the organic content of the organic molecular component of the tissue volume is made by assessment of the organic hydrogen content of the tissue volume.

9. The method of claim 7, wherein
the assessment of the organic content of the tissue volume is made by 13C MRI.

10. The method of claim 1, wherein
the assessment of hydroxyapatite content is made by MRI.

11. The method of claim 10, wherein
hydroxyapatite content is assessed by measuring phosphorus-31 MRI as a proxy for hydroxyapatite.

12. The method of claim 1, wherein
hydroxyapatite content of the tissue volume is estimated by measurement of the calcium content.

13. The method of claim 12, wherein
calcium content is measured by CT scan.

14. The method of claim 1, wherein
the assessment of water content of the tissue volume, the assessment of the organic content of the tissue volume, and the assessment of the hydroxyapatite content of the tissue volume are performed by MRI.

15. The method of claim 1, wherein
Iₘ is calculated as
ln(Iᵥₒₓₑₗ)≈(w_{H}_{₂}_{O} ln(I_{H}_{₂}_{O}))+(w_{org,total}[ln A+Bhᵥₒₓₑₗ^{org}])+(w_{HA} ln(I_{HA})) s.t.Σᵢwᵢ=1
wherein w_{H}_{₂}_{O} is tissue water percentage by mass, is the sum total fractional mass of all organic molecules, A and B are constants that relate hydrogen content in organic molecules to I_{org}, and hᵥₒₓₑₗ^{org} is the total organic molecule hydrogen density by fractional mass in the tissue volume.

16. The method of claim 15, wherein
A is 93.23 eV±10% and B is −3.47±10%.

17. The method of claim 1, wherein
tissue water content and the organic content of the tissue volume are assessed by performing a proton-density weighted scan and a water/organic ¹H separation scan.

18. The method of claim 17, wherein
w_{H}_{₂}_{O}/w_{org,total} and h_{org} are derived from signal data, S, and wherein S is acquired as S ∝ ρ_{H}(1−exp(−TR/T₁))exp(−TE/T₂)
wherein ρ_{H} is the voxel total ¹H content, TR is the repetition time of the pulse sequence, T₁ is the longitudinal relaxation time of the ¹H, TE is the echo time of the pulse sequence, and T₂ is the spin-spin relation time of the ¹H and wherein TR is assumed to be >>T₁ and TE is assumed to be <<T₂.

19. The method of claim 1 wherein
the tissue volume comprises a non-mineralized tissue and the hydroxyapatite content of the tissue volume is assumed to be zero.

21-27. (canceled)
